(19) United States
(12) Patent Application Publication
(10) Pub. No.: US 2002/0076058 A1
(43) Pub. Date: Jun. 20, 2002

(54) Engine rotation reference signal for noise attenuation

(76) Inventor: John Frank Astorino, Livonia, MI (US)

Correspondence Address: LAURA M. SLENZAK, SIEMENS CORPORATION, 186 WOOD AVENUE SOUTH, ISELIN, NJ, 08830, US

(21) Appl. No.: 09/864,673

(22) Filed: May 24, 2001

Related U.S. Application Data
(60) Provisional application No. 60/256,837, filed on Dec. 19, 2000.

(51) Int. Cl. A61F 11/06; G10K 11/16; H03B 29/00

(52) U.S. Cl. 381/71.4; 381/71.5; 381/86

(57) ABSTRACT

The air induction system comprises an air induction body and speaker supported about the air induction body. A control unit communicates with the speaker, controlling its output. The control unit also receives a reference signal from an alternator of a vehicle engine and uses this signal to generate a noise attenuating signal based on the reference signal.

This application claims priority to Provisional Patent Application Serial No. 60/256,837 filed Dec. 19, 2000.

### BACKGROUND OF THE INVENTION

This invention relates to a method and system for noise attenuation.

Vehicle manufacturers have employed active and passive methods to reduce engine noise within the passenger compartment. Such noise frequently emanates from the engine, travels through the air induction system and emanates out of the mouth of the air intake into the passenger compartment. Efforts have been made to reduce the amount of engine noise traveling through the air induction system. These efforts include the use of both passive devices such as expansion chambers and Helmholtz resonators and active devices involving anti-noise generators.

Active systems use a speaker to create a canceling sound that attenuates engine noise. The sound created is out of phase with the engine noise and combines with this noise to result in its reduction. Generally, this sound is generated in proximity to the mouth of the air induction system. In one such system, a control unit, such as a digital signal processor, obtains data from the vehicle engine, creates a predictive model of engine noise, and thereby generates the appropriate cancellation signal based on the results of this model. This signal is then transmitted to the speaker, which transforms this signal into a canceling sound. Because the control unit may not perfectly model engine noise, an error microphone is placed in proximity to the mouth of the air induction system to determine if engine noise need be further attenuated.

The vehicle engine data is typically obtained from an engine speed sensor, which rotates with the turning of the engine crankshaft. An engine speed sensor is a complex and relatively costly device to manufacture and install. This sensor requires a mechanical interface that matches the gearing of the crankshaft and a transducer to convert information from the mechanical interface to an electric signal. This electric signal is then transmitted to the control unit. A need therefore exists to obtain vehicle engine data simply and inexpensively.

### SUMMARY OF THE INVENTION

The invention relates to a method and system for noise attenuation and comprises an air induction body and speaker supported about the air induction body. A control unit communicates with the speaker and controls its output. Rather than obtain engine speed data from an engine speed sensor such as a tachometer, the control unit obtains this information from the engine alternator, which already rotates with the engine crankshaft. The alternator communicates a reference signal to the control unit, which then uses this information to generate a noise attenuating sound. Thus, no additional speed sensor component is necessary. The existing alternator is preferably used.

The reference signal is an alternating current. A rectifier may be used to rectify the reference signal. The rectification may transform the alternating current into a digital signal. A diode may serve as the rectifier.

The air induction system may also include an error microphone, which communicates with the speaker and the control unit. A throttle position sensor may also provide data to the control unit for noise attenuation. The speaker may be at least partially disposed in the mouth of the air induction body. Hence, one embodiment of the system may comprise, the air induction body, the speaker, a control unit, error microphone, the throttle position sensor, and the alternator.

The method of noise attenuation comprises the steps of receiving a reference signal from the alternator. The reference signal is then communicated to the control unit. The control unit uses this signal to generate a noise attenuating signal based on this reference signal. The reference signal may be rectified to provide a digital signal to the control unit.

The current method and system of noise attenuation provides a simple and cost effective means of obtaining vehicle engine speed data. An existing mechanical interface and transducer is used in place of a complex and relatively expensive electromechanical sensor. Moreover, the method and system may be employed in existing air induction and noise attenuation systems with very little modification.

### BRIEF DESCRIPTION OF THE DRAWINGS

The various features and advantages of this invention will become apparent to those skilled in the art from the following detailed description of the currently preferred embodiment. The drawings that accompany the detailed description can be briefly described as follows:

FIG. 1 shows an embodiment of the invention, including air induction body, speaker, control unit, rectifier, and alternator.

FIG. 2 shows a portion of the embodiment of FIG. 1 including alternator and rectifier.

### DETAILED DESCRIPTION OF THE PREFERRED EMBODIMENT

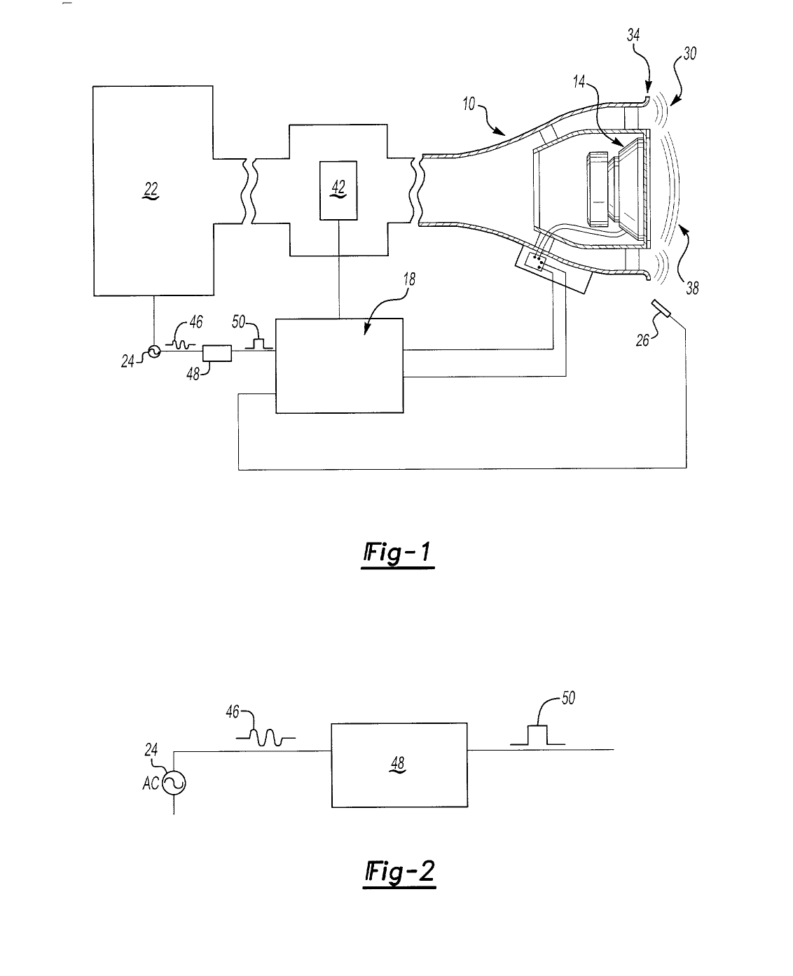

FIG. 1 shows an embodiment of the invention. The air induction system comprises air induction body 10 and speaker 14 supported about air induction body 10. Control unit 18 communicates with speaker 14 and controls its output. Engine noise 30 emanates from engine 22 through air induction body 10 and out mouth 34, which is operatively connected to air induction body 10. As known, control unit obtains data from throttle position sensor 42 to aid in the creation of noise attenuating sound 38 from speaker 14. Typically, noise attenuating sound 38 is out of phase with engine noise 30 to create destructive interference and thereby reduce the volume of both sounds. Error microphone 26 communicates with control unit 18 and serves as feedback to permit further adjustment of noise attenuation.

Rather than employ an engine speed sensor, alternator 24 communicates reference signal 46 to control unit 18. Because alternator 24 is tied to engine 22, alternator 24 provides engine speed data used by control unit 18 for noise attenuation. Reference signal 46 is generally an alternating current, which is rectified by the vehicle's electrical system and used for power immediately or stored by the vehicle battery for later use. The voltage from the alternating current generally ranges from 12 volts to 42 volts. Because the circuitry of control unit 18 operates with digital signals of about 5 volts, reference signal 46 should be obtained prior to rectification by the vehicle electric system and rectified separately to about 5 volts. As seen in FIG. 2, the air induction system includes rectifier 48 to convert the alternating current of reference signal 46 from alternator 24 into digital signal 50 of about 5 volts. Rectifier 48 may be a diode, such as a voltage diode.

The air induction system thus may comprise speaker 14 supported about air induction body 10, control unit 18, error microphone 26, throttle position sensor 42, alternator 24, and rectifier 48. Control unit 18 simply receives reference signal 46 from alternator 24 and thereby generates a noise attenuating signal from the control unit based on reference signal 46, which is preferably converted into digital signal 50 by rectifier 48. In this way, the method and system of noise attenuation provides a simplified manner of obtaining engine data needed for noise attenuation. Alternator 24, an existing mechanical interface and transducer, is used in place of the relatively expensive and complex engine speed sensor. Moreover, very few modifications are consequently necessary to install the system into existing air induction and noise attenuation systems.

The aforementioned description is exemplary rather then limiting. Many modifications and variations of the present invention are possible in light of the above teachings. The preferred embodiments of this invention have been disclosed. However, one of ordinary skill in the art would recognize that certain modifications would come within the scope of this invention. Hence, within the scope of the appended claims, the invention may be practiced otherwise than as specifically described. For this reason the following claims should be studied to determine the true scope and content of this invention.

What is claimed is:

1. An air induction system comprising:
an air induction body;
a speaker supported about said air induction body;
a control unit in communication with said speaker for controlling its output; and
an alternator communicating a reference signal to said control unit.

2. The air induction system of claim 1 wherein said reference signal is an alternating current from said alternator.

3. The air induction system of claim 2 further including a rectifier to rectify said reference signal.

4. The air induction system of claim 3 wherein said rectifier converts said reference signal to a digital signal.

5. The air induction system of claim 4 wherein said rectifier comprises a diode.

6. The air induction system of claim 1 further including an error microphone in communication with said speaker and said control unit.

7. The air induction system of claim 1 further including a throttle position sensor in communication with said control unit.

8. The air induction system of claim 1 further including a mouth operatively connected to said air induction body.

9. The air induction system of claim 1 wherein said speaker is at least partially disposed in said mouth.

10. An air induction system comprising:
an air induction body;
a speaker supported about said air induction body;
a control unit in communication with said speaker for controlling its output;
an error microphone in communication with said speaker and said control unit;
a throttle position sensor in communication with said control unit; and
an alternator communicating a reference signal to said control unit.

11. The air induction system of claim 10 wherein said reference signal is an alternating current from said alternator.

12. The air induction system of claim 111 further including a rectifier to rectify said reference signal.

13. The air induction system of claim 12 wherein said rectifier converts said reference signal to a digital signal.

14. The air induction system of claim 13 wherein said rectifier comprises a diode.

15. The air induction system of claim 10 further including an error microphone in communication with said speaker and said control unit.

16. The air induction system of claim 10 further including a throttle position sensor in communication with said control unit.

17. The air induction system of claim 10 further including a mouth operatively connected to said air induction body.

18. The air induction system of claim 10 wherein said speaker is at least partially disposed in said mouth.

19. A method of noise attenuation comprising the steps of:
receiving a reference signal from an alternator;
communicating the reference signal to a control unit; and
generating a noise attenuating signal from the control unit based on the reference signal.

20. The method of claim 19 further including the step of rectifying the reference signal into a digital signal.
